SEQUENCE LISTING (4 sequences)

SEQ ID NO: 1 - protein, 727 aa, Homo sapiens
MWVTKLLPALLLQHVLLHLLLLPIAIPYAEGHKKRRNTIHEFKKSAKTTLIKIDPALKIKTKKVNTADQCANRCTRNNGLPFTCKAFVFDKARKQCLWFPFNSMSSGVKKEFGHEFDLYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTVNDTDVPMETTECIQGQGEGYRGTANTIWNGIPCQRWDSQYPHKHDMTPENFKCKDLRENYCRNPDGSESPWCFTTDPNIRVGYCSQIPNCDMSNGQDCYRGNGKNYMGNLSQTRSGLTCSMWNKNMEDLHRHIFWEPDASKLNENYCRNPDDDAHGPWCYTGNPLIPWDYCPISRCEGDTTPTIVNLDHPVISCAKTKQLRVVNGIPTRTNVGWMISLRYRNKHICGGSLIKESWVLTARQCFPSRDLKDYEAWLGIHDVHGRGEEKRKQVLNVSQLVYGPEGSDLVLMKLARPAVLDDFVNTIDLPNYGCTIPEKTSCSVYGWGYTGLINYDGLLRVAHLYIMGNEKCSQHHRGKVTLNESEICAGAEKIGSGPCEGDYGGPLVCEQHKMRMVLGVIVPGRGCAIPNRPGIFVRVAYYAKWIHKILTYKVPQS

SEQ ID NO: 2 - protein, 269 aa, Homo sapiens
LYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMS

SEQ ID NO: 3 - protein, 368 aa, Homo sapiens
  MISC_FEATURE (325)..(325): “Xaa” = any amino acid
LYENKDYIRNCIIGKGRSYKGTVSITKSGIKCQPWSSMIPHEHSFLPSSYRGKDLQENYCRNPRGEEGGPWCFTSNPEVRYEVCDIPQCSEVECMTCNGESYRGLMDHTESGKICQRWDHQTPHRHKFLPERYPDKGFDDNYCRNPDGQPRPWCYTLDPHTRWEYCAIKTCADNTMNDTDVPLETTECIQGQGEGYRGTVNTIWNGIPCQRWDSQYPHEHDMTPENFKCKDLRENYCRNPDGSESPWCFTTDPNIRVGYCSQIPNCDMSHGQDCYRGNGKNYMGNLSQTRSGLTCSMWDKNMEDLHRHIFWEPDASKLNENYCRXPDDDAHGPWCYTGNPLIPWDYCPISRCEGDTTPTIVNLDHPVI

SEQ ID NO: 4 - protein, 710 aa, Homo sapiens
MGWLPLLLLLTQCLGVPGQRSPLNDFQVLRGTELQHLLHAVVPGPWQEDVADAEECAGRCGPLMDCRAFHYNVSSHGCQLLPWTQHSPHTRLRRSGRCDLFQKKDYVRTCIMNNGVGYRGTMATTVGGLPCQAWSHKFPNDHKYTPTLRNGLEENFCRNPDGDPGGPWCYTTDPAVRFQSCGIKSCREAACVWCNGEEYRGAVDRTESGRECQRWDLQHPHQHPFEPGKFLDQGLDDNYCRNPDGSERPWCYTTDPQIEREPCDLPRCGSEAQPQEATTVSCFRGKGEGYRGTANTTTAGVPCQRWDAQIPHQHRFTPEKYACKDLRENFCRNPDGSEAPWCFTLRPGMRAAFCYQIRRCTDDVRPQDCYHGAGEQYRGTVSKTRKGVQCQRWSAETPHKPQFTFTSEPHAQLEENFCRNPDGDSHGPWCYTMDPRTPFDYCALRRCADDQPPSILDPPDQVQFEKCGKRVDRLDQRRSKLRVVGGHPGNSPWTVSLRNRQGQHFCGGSLVKEQWILTARQCFSSCHMPLTGYEVWLGTLFQNPQHGEPSLQRVPVAKMVCGPSGSQLVLLKLERSVTLNQRVALICLPPEWYVVPPGTKCEIAGWGETKGTGNDTVLNVAFLNVISNQECNIKHRGRVRESEMCTEGLLAPVGACEGDYGGPLACFTHNCWVLEGIIIPNRVCARSRWPAVFTRVSVFDWIKHKVMRLG

(19) United States
(12) Patent Application Publication
(10) Pub. No.: US 2004/0121945 A1
(43) Pub. Date: Jun. 24, 2004

(54) Compositions and methods for inhibiting endothelial cell proliferation

(76) Inventors: Hong Liang, Gaithersburg, MD (US); Kim Lee Sim, Gaithersburg, MD (US); Clara Dey, Germantown, MD (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/149,103

(22) PCT Filed: Dec. 15, 2000
(86) PCT No.: PCT/US00/34039

(51) Int. Cl. A61K 38/18

(52) U.S. Cl. 514/12

(57) ABSTRACT

Compositions and methods for regulating angiogenic activity wherein the compositions comprise proteins belonging to the family of kringle domain containing proteins and peptides and active fragments thereof are provided. More specifically, compositions and methods comprising kringle domain containing proteins and peptides such as hepatocyte growth factor (HGF) and/or macrophage stimulating protein (MSP), and biologically active fragments thereof are provided. HGF protein fragments of the present invention exhibit potent antiangiogenic activity on human and other animal cells, particularly endothelial cells. More particularly, compositions comprising HGF fragments, and/or HGF fragment homologs, may be combined with a pharmaceutically acceptable excipient or carrier and used to inhibit angiogenesis and angiogenesis-related diseases such as cancer, arthritis, macular degeneration, and diabetic retinopathy.

### TECHNICAL FIELD

This application relates to novel uses for kringle domain-containing proteins and peptides such as hepatocyte growth factor (HGF), also known as scatter factor, and macrophage stimulating protein (MSP), as regulators of angiogenesis useful for treating angiogenesis-related diseases including angiogenesis-dependent cancer. More specifically, the present invention relates to kringle domain containing proteins and peptides including active fragments of HGF and/or MSP, capable of regulating, and preferably inhibiting angiogenesis. The invention further relates to novel HGF and/or MSP fragment compositions and methods for curing angiogenesis-dependent disorders such as cancer, arthritis, blindness, diabetic retinopathy, macular degeneration, psoriasis and artherosclerosis. In addition, the present invention relates to kringle domain containing proteins and peptides antibodies that, for example, block the interaction of HGF (and fragments thereof) to the c-met receptor, the molecular probes for monitoring biosynthesis to antibodies that are specific for kringle domain containing proteins and peptides, to the development of peptide agonists and antagonists to kringle domain containing proteins and peptides, and to cytotoxic agents linked to receptors peptides.

### BACKGROUND OF THE INVENTION

Kringle domain containing proteins and peptides are unique in that they are characterized by triple disulfide loops structures. Examples of such proteins comprise hepatocyte growth factor, macrophage stimulating protein, tissue plasminogen activator, apolipoprotein (a), prothrombin, urokinase, and ANGIOSTATIN proteins.

Hepatocyte growth factor (HGF) is a mesenchyme derived glycoprotein and is named for its ability to induce kidney epithelial cells in a collagen matrix to form branching networks of tubules. (Grant et al. PNAS 1993; 90:1937-1941) As characterized in the art, HGF is considered to be a potent angiogenic molecule that primarily acts on endothelial cells promoting cell motility, proliferation, protease production, invasion and organization into capillary-like tubules. (Rosen et al. Adv. Cancer Res. 1995; 67:257-79) HGF contains 29% identity with plasminogen and within the first four kringle domains HGF contains 44% similarity with plasminogen. HGF contains approximately 44% identity with macrophage stimulating protein (MSP). In addition, all of the cystenes and most of the aromatic amino acids such as trytophanes and prolines are conserved between HGF and plasminogen. It is produced as a preproprecursor of 728 amino acids that is cleaved to a large heterodimeric molecule made up of an α-chain (69 kDa) and a β-chain (34 kDa). (Lokker et al. Prot. Engin., 1994; 7:895-903) HGF is a basic heparin-binding glycoprotein consisting of a heavy (58 kDa) and a light (31 kDa) subunit. (Grant et al.) The biological effects of HGF are triggered by the interaction of HGF with its high-affinity receptor c-Met. The c-Met is a receptor-type tyrosine kinase containing a 145 kDa β-chain that transverses the membrane once and an extracellular 50 kDa α-chain. HGF-Met signaling has been implicated in supporting significant roles in the pathogenesis and biology of human cancers. Specifically it is thought that by autocrine or paracrine mechanisms HGF-Met signaling promotes tumor cell growth, invasion and angiogenesis.

As mentioned above, the structure of HGF is similar to that of plasminogen. The α-chain is distinguished by the presence of an N-terminal hairpin loop followed by four kringle domains, and the β-chain by a non-functional serine protease-like domain. Structural-functional studies have demonstrated that the hairpin loop and kringle domains are important in the binding of HGF to its receptors and proteoglycans. (To et al. Oncl. Rep., 1998; 5:1013-1024)

Macrophage Stimulating Protein

Macrophage stimulating protein (MSP) is a 78 KD plasma protein (711aa) that is secreted by the liver into the circulation as single-chain, biologically inactive pro-MSP. After the proteolytic cleavage at a single site MSP becomes biologically active disulfide-linked alpha beta-chain heterodimeric molecule. MSP is a growth and motility factor which interacts to its transmembrane tyrosine kinase called RON to induce activation of signal transduction pathway that mediates its biological effects.

Both MSP and HGF are plasminogen-related growth and motility factors that interact with cell-surface protein tyrosine kinase receptors. Each one is a heterodimeric protein comprising a disulfide-linked alpha chain and a serine protease-like beta chain. Despite structural similarities between MSP and HGF, the primary receptor binding site is located on the alpha chain of HGF but on the beta chain of MSP.

Evaluation studies indicated that HGF and HGF1/MSP evolved along with plasminogen and other members of the kringle-serine proteinase superfamily from an ancestral gene that contained a single copy of the kringle domain, a serine protease domain and an activation peptide connecting the two domains. So the kringle domains from plasminogen, HGF and HGF1/MSP may still possess similar biological functions such as anti-angiogenesis.

Angiogenesis and Cancer

The hypothesis that tumor growth is angiogenesis-dependent was first proposed in 1971 by Judah Folkman (N. Engl. Jour. Med. 285:1182 1186, 1971). In its simplest terms the hypothesis proposes that expansion of tumor volume beyond a certain phase requires the induction of new capillary blood vessels. For example, pulmonary micrometastases in the early prevascular phase in mice would be undetectable except by high power microscopy on histological sections. Further indirect evidence supporting the concept that tumor growth is angiogenesis dependent is found in U.S. Pat. Nos. 5,639,725, 5,629,327, 5,792,845, 5,733,876, and 5,854,205, all of which are incorporated herein by reference.

To stimulate angiogenesis, tumors upregulate their production of a variety of angiogenic factors, including the fibroblast growth factors (aFGF and bFGF) (Kandel et al., 1991) and vascular endothelial cell growth factor/vascular permeability factor (VEGF/VPF) and HGF. However, many malignant tumors also generate inhibitors of angiogenesis, including ANGIOSTATIN protein and thrombospondin. (Chen et al., 1995; Good et al., 1990; O'Reilly et al., 1994). It is postulated that the angiogenic phenotype is the result of a net balance between these positive and negative regulators of neovascularization. (Good et al., 1990; O'Reilly et al., 1994; Parangi et al., 1996; Rastinejad et al., 1989). Several other endogenous inhibitors of angiogenesis have been identified, although not all are associated with the presence of a tumor. These include, platelet factor 4 (Gupta et al., 1995; Maione et al., 1990), interferon-alpha, interferon-inducible protein 10 (Angiolillo et al., 1995; Strieter et al., 1995), which is induced by interleukin-12 and/or interferon-gamma (Voest et al., 1995), gro-beta (Cao et al., 1995), and the 16 kDa N-terminal fragment of prolactin (Clapp et al., 1993).

One example of an angiogenesis inhibitor that specifically inhibits endothelial cell proliferation is ANGIOSTATIN protein. (O'Reilly et al., 1994). ANGIOSTATIN protein is an approximately 38 kiloDalton (kDa) specific inhibitor of endothelial cell proliferation. ANGIOSTATIN protein is an internal fragment of plasminogen containing at least three of the five kringles of plasminogen. ANGIOSTATIN protein has been shown to reduce tumor weight and to inhibit metastasis in certain tumor models. (O'Reilly et al., 1994). Another angiogenesis inhibitor is ENDOSTATIN protein, which is a carboxy fragment of collagen or XVIII. (O'Reilly et al., 1997).

What is needed is the discovery and development of additional anti-angiogenic agents that may be used alone or in combination with known angiogenic agents in order to treat cancer and hyperproliferative disorders.

### SUMMARY OF THE INVENTION

The present invention comprises novel uses for kringle domain-containing proteins and peptides such as hepatocyte growth factor (HGF), also known as scatter factor, and macrophage stimulating protein (MSP), as regulators of angiogenesis useful for treating angiogenesis-related diseases including angiogenesis-dependent cancer. More specifically, the present invention relates to kringle domain containing proteins and peptides including active fragments of HGF and/or MSP, capable of regulating, and preferably inhibiting angiogenesis. The invention further relates to novel HGF and/or MSP fragment compositions and methods for curing angiogenesis-dependent disorders such as cancer, arthritis, blindness, diabetic retinopathy, macular degeneration, psoriasis and artherosclerosis. In addition, the present invention relates to kringle domain containing proteins and peptides antibodies that, for example, block the interaction of HGF (and fragments thereof) to the c-met receptor, the molecular probes for monitoring biosynthesis to antibodies that are specific for kringle domain containing proteins and peptides, to the development of peptide agonists and antagonists to kringle domain containing proteins and peptides, and to cytotoxic agents linked to receptors peptides. The present invention generally relates to kringle domain containing proteins and peptides and active fragments thereof, as angiogenesis inhibitors and methods of use thereof. Examples of such proteins include hepatocyte growth factor (HGF), macrophage stimulating protein (MSP), apolipoprotein, prothrombin urokinase and tissue plasminogen activator. Whole HGF is a potent and specific regulator of endothelial cell function and angiogenesis, however, as demonstrated herein, HGF fragments comprising kringle domains serve the reverse effects of whole HGF. Systemic therapy with active HGF fragments, causes suppression of tumor-induced angiogenesis, and exhibits strong antitumor activity.

HGF, also known as scatter factor, has a molecular weight of approximately 87 kiloDaltons as determined by amino acid composition. As described herein, novel HGF fragments are capable of inhibiting endothelial cell function in cultured endothelial cells, tumor cells, smooth muscle cells, and other variety of cells.

The present invention provides methods and compositions for treating diseases and processes mediated by undesired and uncontrolled angiogenesis by administering to a human or animal with the undesired angiogenesis a composition comprising kringle domain containing proteins and peptides such as novel HGF and/or MSP active fragments of the present invention, or derivatives thereof, in a dosage sufficient to regulate, and preferably inhibit, angiogenesis. More specifically, the present invention is directed kringles 1-3, and/or kringles 2-3 of HGF and/or MSP. The present invention is particularly useful for treating or for repressing the growth of tumors. Administration of the presently identified novel HGF and/or MSP fragments to a human or animal with metastasized tumors prevents the growth or expansion of those tumors. The invention further provides methods and compositions for regulating endothelial cell function in vivo as well as in vitro.

The present invention also includes kringle domain-containing proteins and peptides such as HGF peptide fragments that can be labeled isotopically or with other molecules or proteins for use in the detection and visualization of HGF binding sites with state of the art techniques, including, but not limited to, positron emission tomography, autoradiography, flow cytometry, radioreceptor binding assays, and immunohistochemistry.

The present invention also includes kringle domain containing proteins and peptides, kringle domain containing proteins and peptides fragments, or kringle domain containing proteins and peptides receptor agonists and antagonists linked to cytotoxic agents for therapeutic and research applications.

The present invention also includes HGF, HGF fragments, or HGF receptor agonists and antagonists linked to cytotoxic agents for therapeutic and research applications.

The present invention also includes MSP, MSP fragments, or MSP receptor agonists and antagonists linked to cytotoxic agents for therapeutic and research applications.

In addition, kringle domain containing proteins and peptides may act as agonists and antagonists of kringle domain containing protein receptors, thereby enhancing or blocking the biological activity of such proteins. Such proteins and peptides are used in the isolation of the receptors such as the HGF receptor.

A surprising discovery is that various active fragments of HGF, can serve as sustained release anti-angiogenesis compounds when administered to a tumor-bearing animal.

The present invention also relates to methods of using kringle domain containing proteins and peptides and fragments thereof, corresponding nucleic acid sequences, and antibodies that bind specifically to the inhibitor and its peptides, to diagnose endothelial cell-related diseases and disorders.

The invention further encompasses a method for identifying receptors specific HGF fragments, and the receptor molecules identified and isolated thereby.

An important medical method is a new form of birth control, wherein an effective amount of a kringle domain from a kringle domain containing protein such as HGF or MSP is administered to a female such that uterine endometrial vascularization is inhibited and embryo implantation cannot occur or be sustained.

A particularly important aspect of the present invention is the discovery of a novel and effective method for treating angiogenesis-related diseases, particularly angiogenesis-dependent cancer, in patients, and for curing angiogenesis-dependent cancer in patients. The method unexpectedly provides the medically important result of inhibition of tumor growth and reduction of tumor mass. The method relates to the co-administration of an active HGF (or MSP) fragment of the present invention and another anti-angiogenesis compound, such as ANGIOSTATIN protein (EntreMed, Inc. Rockville, Md.) or ENDOSTATIN protein (EntreMed, Inc. Rockville, Md.). Accordingly, the present invention also includes formulations containing HGF kringle fragments, MSP kringle fragments, ANGIOSTATIN protein, and/or ENDOSTATIN protein, which are effective for treating or curing angiogenesis-dependent diseases.

Accordingly, it is an object of the present invention to provide compositions and methods comprising kringle domain containing proteins and peptides such as HGF, including active HGF fragments, useful for the treatment of angiogenic disorders.

Another object of the present invention is to provide compositions and methods comprising kringle domain containing proteins and peptides, or kringle domain containing proteins and peptides fragments in combination with other antiangiogenic compounds such as ANGIOSTATIN protein or ENDOSTATIN™ protein, useful for the treatment of angiogenic disorders.

Another object of the present invention is to provide compositions and methods comprising HGF, or HGF kringle domain containing fragments in combination with other antiangiogenic compounds such as ANGIOSTATIN protein or ENDOSTATIN™ protein, useful for the treatment of angiogenic disorders.

Another object of the present invention is to provide compositions and methods comprising MSP, or MSP kringle domain containing fragments, in combination with other antiangiogenic compounds such as ANGIOSTATIN protein or ENDOSTATIN protein, useful for the treatment of angiogenic disorders.

It is another object of the present invention to provide compositions and methods of treating diseases and processes that are mediated by angiogenesis.

It is yet another object of the present invention to provide compositions and methods for treating diseases and processes that are mediated by angiogenesis including, but not limited to, hemangioma, solid tumors, leukemia, metastasis, telangiectasia psoriasis scleroderma, pyogenic granuloma, myocardial angiogenesis, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovascularization, macular degeneration, wound healing, surgical adhesions, peptic ulcer, fractures, keloids, vasculogenesis, hematopoiesis, ovulation, menstruation, and placentation.

It is another object of the present invention to provide compositions and methods for treating or repressing the growth of a cancer.

Still another object of the present invention is to provide compositions and methods comprising antibodies to kringle domain containing proteins and peptides, or kringle domain containing proteins and peptides fragments, that are selective for specific regions of the kringle domain containing proteins and peptides molecule.

Another object of the present invention is to provide compositions and methods comprising antibodies to HGF, or HGF fragments, that are selective for specific regions of the HGF molecule.

Still another object of the present invention is to provide compositions and methods comprising antibodies to MSP, or MSP fragments, that are selective for specific regions of the MSP molecule.

It is another object of the present invention to provide compositions and methods for the detection or prognosis of anti-angiogenesis activity.

It is yet another object of the present invention to provide a therapy for cancer that has minimal side effects.

Still another object of the present invention is to provide compositions comprising kringle domain containing proteins and peptides such as HGF or HGF peptide fragments, linked to a cytotoxic agent for treating or repressing the growth of a cancer.

These and other objects, features and advantages of the present invention will become apparent after a review of the following detailed description of the disclosed embodiments and the appended claims.

### BRIEF DESCRIPTION OF THE FIGURES

FIG. 1 shows a structural comparison of HGF and plasminogen. Mature HGF is a heterodimeric molecule which is composed of an alpha-chain, containing the N-terminal hairpin domain and four kringle domains, and a beta-chain containing the serine protease-like domain. The alpha-chain has been identified as the receptor binding domain. HGF/NK1, HGF/NK2, HGF/NK3 and HGF/NK4 are defined as N-terminal hairpin domain plus the first kringle (NK1), or N-terminal hairpin domain plus the first 2 kringles (NK2) and so on HGF K2-3 comprises the second and the third kringles of HGF. Plasminogen is composed of kringle domains and serine protease domain. Angiostatin protein comprises the first 3 kringles of plasminogen (K1-3).

FIG. 2 shows protein sequence comparison between kringles 1-4 of HGF and plasminogen. Primary protein sequence comparison revealed that plasminogen kringle domain and HGF kringle domain has over 49% sequence similarity and all the cysteines are highly conserved.

FIG. 3 provides the amino acid sequence for HGF (SEQ ID NO: 1).

FIG. 4 provides the amino acid sequence for HGF fragments (SEQ ID NOS:2 and 3).

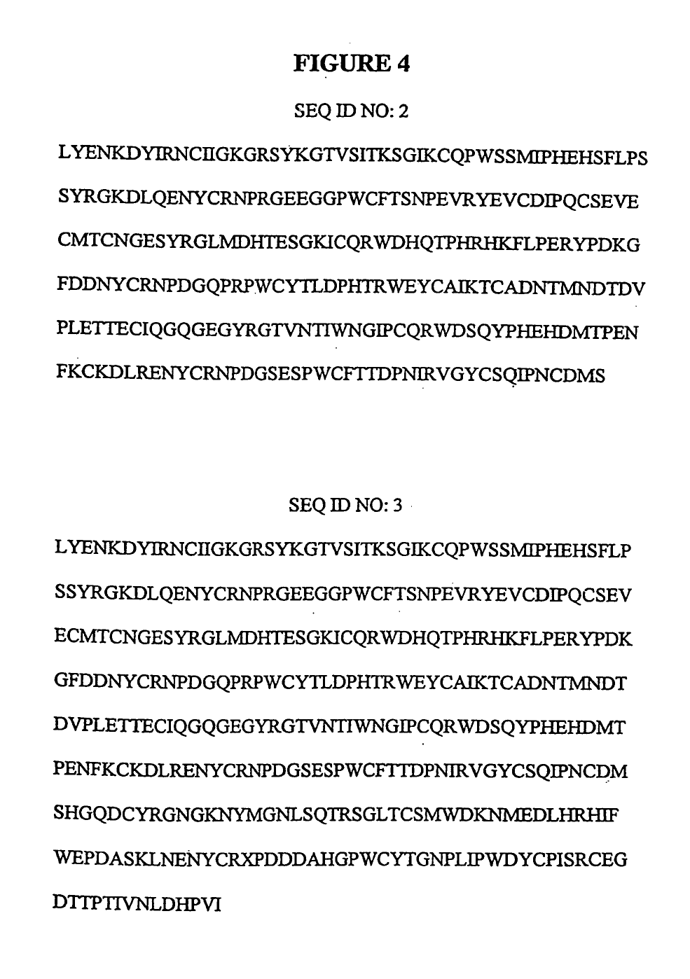

FIG. 5 provides the amino acid sequence for MSP (SEQ ID NO: 4).

FIG. 6 is a SDS-PAGE gel of recombinant HGF K2-3 expressed in Pichia pastori.

FIGS. 7(a) and 7(b) are graphs showing the HGF K2-3 inhibits VEGF as well as HGF stimulated HUVEC migration.

FIG. 8 is a graph showing HGF K2-3 inhibits HUVEC tube formation.

FIG. 9 is a graph showing that Angiostatin protein inhibits HGF stimulated HUVEC migration.

FIG. 10 shows that Angiostatin protein does not inhibit HGF stimulated c-met phosphorylation.

FIG. 11 shows Western blots probed with anti-phosphotyrosine antibody (4G10) or anti-c-met antibodies.

### DETAILED DESCRIPTION OF THE INVENTION

The present invention may be understood more readily by reference to the following detailed description of specific embodiments included herein. Although the present invention has been described with reference to specific details of certain embodiments thereof, it is not intended that such details should be regarded as limitations upon the scope of the invention. The entire text of the references mentioned herein are hereby incorporated in their entireties by reference.

Applicants have discovered a novel property for a class of protein molecules. These protein molecules are characterized in that they typically include kringle domains, and/or protease domains, and have the surprising ability to regulate angiogenic function when added to proliferating endothelial cells. “Hepatocyte Growth Factor” (HGF) and macrophage stimulating protein (MSP) along with tissue plasminogen activator, apolipoprotein (a), prothrombin and urokinase, are examples of such proteins. HGF and MSP belong to the plasminogen-related kringle domain family, and as used herein, it is to be understood that the term HGF includes HGF analogs, homologs, HGF kringle domain fragments and active peptides thereof, and that the term MSP includes MSP analogs, homologs, and active peptides thereof.

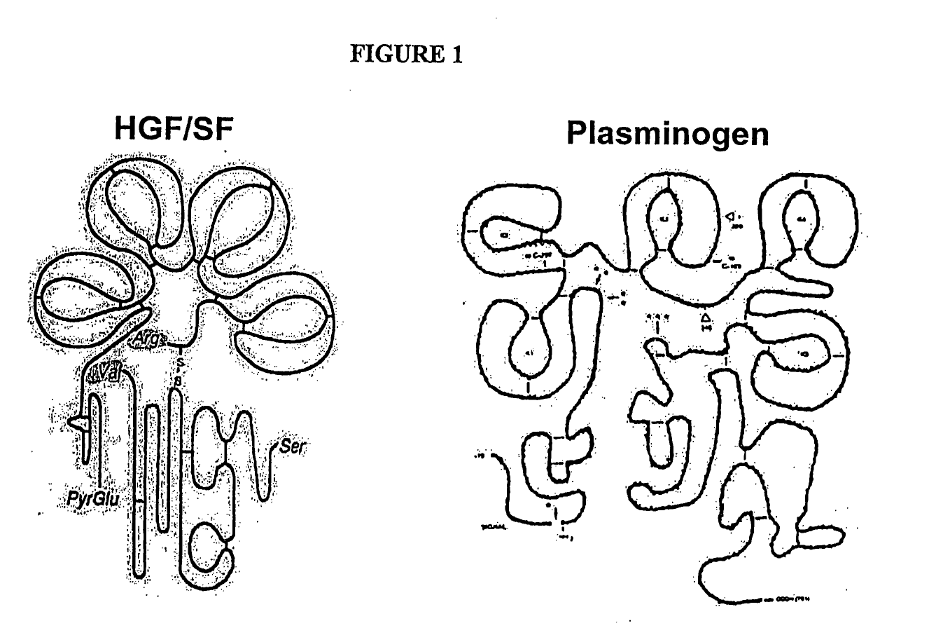

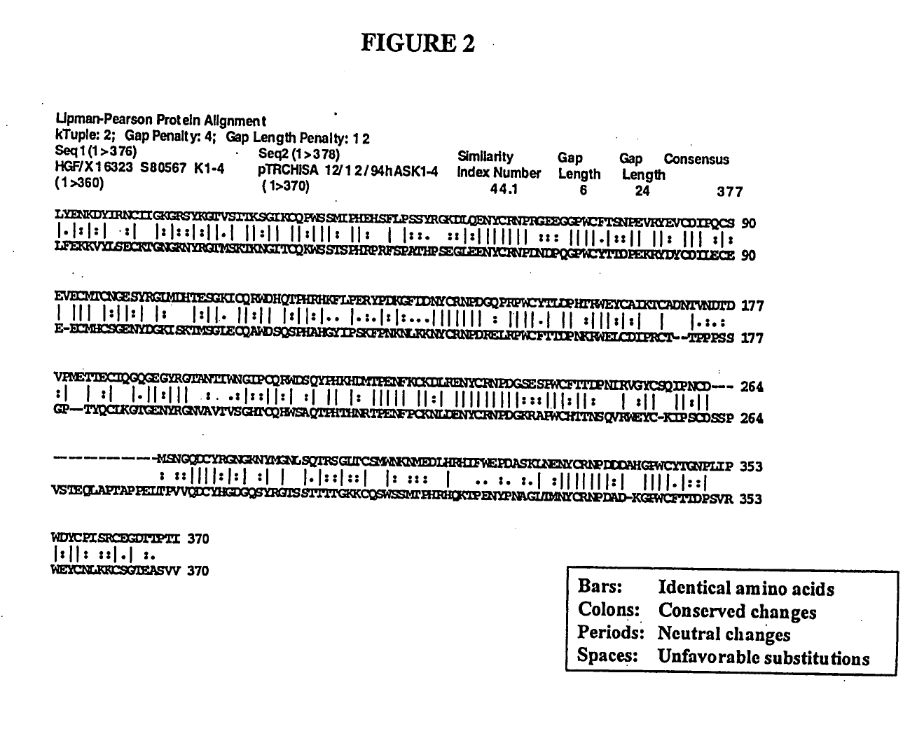

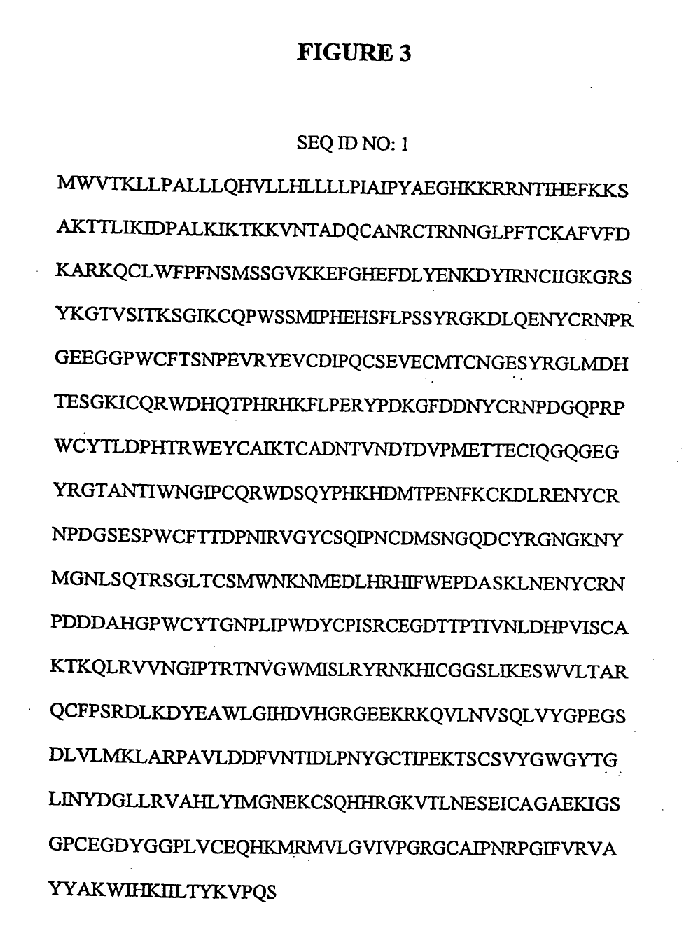

The term “Hepatocyte Growth Factor” (HGF) refers generally to a protein that is approximately 87 kiloDaltons in size as determined by amino acid composition, more specifically to a protein that is approximately 43 kiloDaltons, and more preferably to a protein that is approximately 31 kiloDaltons. HGF shares both structural (FIG. 1) and sequence homology (FIG. 2) with plasminogen. The amino acid sequence of a human HGF is provided in SEQ ID. NO: 1 as shown in FIG. 3. The term HGF also includes precursor forms of the prepropeptide and propeptide as well as modified proteins and peptides that have a substantially similar amino acid sequence, and which are capable of inhibiting proliferation of endothelial cells. For example, silent substitutions of amino acids, wherein the replacement of an amino acid with a structurally or chemically similar amino acid does not significantly alter the structure, conformation or activity of the protein, are well known in the art.

Such silent substitutions, additions and deletions, are intended to fall within the scope of the appended claims.

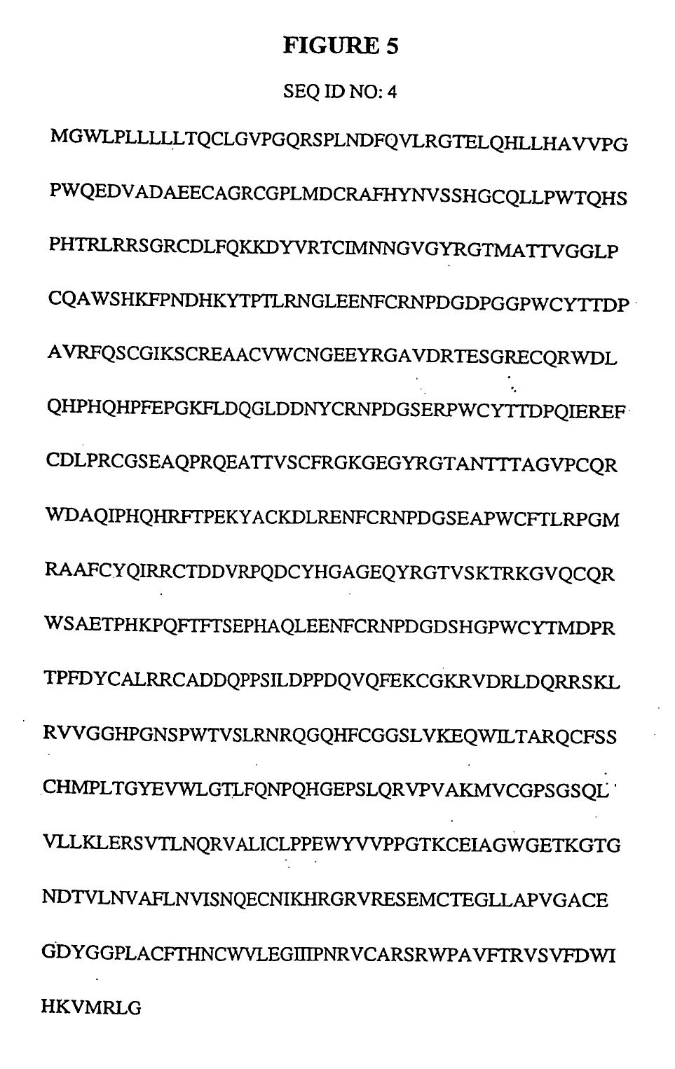

The term “Macrophage Stimulating Protein” (MSP) refers generally to a protein that is approximately 78 kilodaltons in size as determined by amino acid composition, and shares both structural and sequence homology with plasminogen. The amino acid sequence of a human HGF is provided in SEQ ID. NO: 4 as shown in FIG. 5. The term MSP also includes precursor forms of the prepropeptide and propeptide as well as modified proteins and peptides that have a substantially similar amino acid sequence, and which are capable of inhibiting proliferation of endothelial cells. For example, silent substitutions of amino acids, wherein the replacement of an amino acid with a structurally or chemically similar amino acid does not significantly alter the structure, conformation or activity of the protein, are well known in the art. Such silent substitutions, additions and deletions, are intended to fall within the scope of the appended claims.

It will be appreciated that the terms “HGF” and “MSP” include shortened proteins or peptides wherein one or more amino acid is removed from either or both ends of the protein, or from an internal region of the protein, yet the resulting molecule retains angiogenic regulating activity. HGF and MSP also include lengthened proteins or peptides wherein one or more amino acid is added to either or both ends of the protein, or to an internal location in the protein, yet the resulting molecule retains angiogenic regulating activity. Such molecules, for example with tyrosine added in the first position, are useful for labeling such as radioiodination with ¹²⁵Iodine, for use in assays. Labeling with other radioisotopes may be useful in providing a molecular tool for isolating and identifying the target cell containing HGF of MSP receptors. Other labeling, with molecules such as ricin, may provide a mechanism for destroying cells with HGF or MSP receptors. The invention also contemplates that active peptides of HGF may be used alone or combined with other peptides and proteins to form chimeric proteins containing active HGF or MSP peptides. Active HGF fragments of particular interest include kringles 1-3 or HGF as set forth in SEQ ID NO:2 (269 amino acids, 31 kiloDaltons) and kringles 1-4 of HGF as set forth in SEQ ID NO:3 (368 amino acids, 43 kiloDaltons). Both SEQ ID NOS: 2 and 3 were obtained from the Pichia production clone.

“Substantial sequence homology” means at least approximately 70% homology between amino acid residue sequence in the kringle domain containing protein analog, homolog or derivative sequence and that of kringle domain containing protein, preferably at least approximately 80% homology, and more preferably at least approximately 90% homology.

Kringle domain containing proteins and peptides such as HGF can be isolated from normal, hyperplastic, primary and metatstatic tissue from a variety of species including humans. HGF and MSP can also be isolated from body fluids including, but not limited to, semen, serum, urine and ascites, or synthesized by chemical or biological methods (e.g. peptide synthesis and in vitro enzymatic catalysis of precursor molecules to yield active HGF). Kringle domain containing proteins and peptides may be produced from recombinant sources, from genetically altered cells implanted into animals, from tumors, and from cell cultures as well as other sources. Recombinant techniques include gene amplification from DNA sources using the polymerase chain reaction (PCR), and gene amplification from RNA sources using reverse transcriptase/PCR.

Though not wishing to be bound by the following theory, fragments of glycoproteins in the family of kringle domain containing proteins and peptides such as HFG or MSP regulate angiogenic activity by specifically, and most likely reversibly, inhibiting endothelial cell proliferation. The inhibitor protein molecules of the present invention are useful as birth control drugs, and for treating angiogenesis-related diseases, particularly angiogenesis-dependent cancers and tumors. The protein molecules are also useful for curing angiogenesis-dependent cancers and tumors. The unexpected and surprising ability of these novel compounds to treat and cure angiogenesis-dependent cancers and tumors answers a long-felt, unfulfilled need in the medical arts, and provides an important benefit to mankind.

Important terms that are used herein are defined as follows. “Cancer” means angiogenesis-dependent cancers and tumors, i.e. tumors that require for their growth (expansion in volume and/or mass) an increase in the number and density of the blood vessels supplying them with blood. “Regression” refers to the reduction of tumor mass and size.

As used herein, the term “angiogenesis” and related terms such as “angiogenic” refer to activities associated with blood vessel growth and development, including, but not limited to, endothelial cell proliferation, endothelial cell migration and capillary tube formation.

As used herein, the term “antiangiogenic” refers to compositions and the like that are capable of inhibiting the formation of blood vessels, including but not limited to inhibiting endothelial cell proliferation, endothelial cell migration and capillary tube formation.

The process of angiogenesis is complex and involves a number of orchestrated steps that can be separately studied in vitro, such as FGF-2- and/or VEGF-stimulated endothelial cell proliferation and migration. For example, ANGIOSTATIN protein and ENDOSTATIN protein inhibit these processes (see U.S. Pat. No. 5,639,725 and U.S. Pat. No. 5,854,205). The inventors of the present invention have suprisingly discovered antiangiogenic properties of proteins belonging to the family of kringle domain containing proteins and peptides by demonstrating and systematically evaluating the effects of such proteins, for example HGF, on endothelial cell proliferation, migration, and invasion.

Contrary to the teachings of the prior art wherein HGF is characterized as “potent angiogenic molecule, primarily act[ing] on endothelial cells inducing cell motility, proliferation, protease production, invasion, and organization into capillary-like tubes” (Rosen et al. Adv. Cancer Res. 1995; 67:257-79), the inventors of the present invention demonstrate the opposite effect of novel HGF fragments as an anti-angiogenic molecule. Until the discovery of the novel HGF fragments as described herein, HGF was considered to be only related to the induction of angiogenesis: “HGF/SF has been shown to stimulate endothelial cell proliferation and migration, and induce angiogenesis in vivo. HGF/SF may also potentiate new blood vessel formation by up-regulating the expression of vascular endothelial cell growth factor (VEGF) in vascular smooth muscle cells.” (To et al. 1998) Accordingly the prior art in fact teaches away from the study of HGF related proteins or HGF fragments as exhibiting any angiogenic activity.

The effects of the novel HGF fragments of the present invention on angiogenic activity are demonstrated in Human Umbilical Vein Endothelial Cells (HUVEC). Purified human HGF fragments demonstrate a potent and dose related inhibitory activity on FGF-2-stimulated proliferation of HUVEC cells. To determine if HGF fragments inhibit a variety of endothelial cells or simply display specificity for HUVECs, the ability of HGF to inhibit bovine adrenal cortex endothelial cell (BCE) and human microvascular dermal cell (HMVEC-d) proliferation is also demonstrated. The effects of HGF on FGF-2-stimulated endothelial cell proliferation are also conducted.

In order to demonstrate that HGF fragments exert antiangiogenic effects as opposed to general inhibition of cell proliferation, the inventors demonstrate experiments to show direct stimulatory or inhibitory effect on the proliferation of cancer cells.

The effects of HGF fragments on endothelial cell migration are demonstrated by the inventors to further confirm the antiangiogenic effects of such fragments. In order to evaluate the in vitro effects of HGF fragments on endothelial cell migration in response to FGF-2 or VEGF, confluent monolayers of HUVEC are scraped to remove a section of monolayer and cultured with FGF-2 or VEGF in the presence or absence of purified human HGF fragments.

The inventors further demonstrate antiangiogenic properties of HGF fragments by demonstrating effects on endothelial cell invasion. These experiments demonstrate that inhibition is dose dependent and not the result of toxicity as the endothelial cells appear viable and no junctions are made by the cells. These findings further support the inhibitory effects of HGF fragments on endothelial cell invasion and further confirm HGF fragment antiangiogenic activity.

Further studies are conducted to determine whether HGF fragments of the present invention function similarly to other kringle domain containing proteins such as ANGIOSTATIN protein. For examples the inventors are conducting experiments to determine whether ANGIOSTATIN protein binds the HGF receptor c-met and acts as an antagonist to compete with HGF binding, thus inhibiting tumorgenesis. The inventors are using a tumor cell line with expresses c-met receptor to determine whether ANGIOSTATIN protein blocks downstream effects (cell migration, proliferation, morphology, cytokine production level, phosphorylations, etc.) of HGF.

Corresponding experiments described above for HGF fragments, are also conducted for MSP.

Protocols and methods for conducting the above-described experiments are well-known to those skilled in the art and are described in further detail in the Examples below, and in U.S. patent application Ser. No. 09/316,802.

Though not wishing to be bound by the following theory, it is believed that the antiangiogenic properties of the present HGF fragments are related to the kringle activity of the protein. More specifically it is believed that the antiangiogenic activity is most likely located within kringles 1-3 (SEQ ID NO:2) or within kringles 1-4 (SEQ ID NO:3). It is also believed that the antiangiogenic properties of the MSP and fragments thereof are related to the kringle activity of the protein and that such antiangiogenic activity is located within kringles 1-3 or within kringles 1-4 of MSP.

In conducting the above-described experiments, the inventors of the present invention suprisingly demonstrate for the first time that certain novel HGF fragments are endothelial cell-specific inhibitors of angiogenesis that exhibit potent anti-proliferative and anti-migratory activity on a variety of cultured endothelial cells. Furthermore, these novel HGF fragments inhibit the endothelial-cell specific angiogenesis process of capillary tube formation in matrigel.

Based on the novel findings of the inventors, the present invention is directed to methods and compositions comprising the administration of proteins belonging to the kringle domain containing protein family for the regulation of antiangiogenic processes. More particularly, the methods and compositions of the present invention comprise the administration of novel HGF or MSP fragments for inhibiting angiogenesis and for reducing related cancer or tumor growth.

The novel antiangiogenic HGF or MSP fragments of the present invention can be made by automated protein synthesis methodologies well-known to one skilled in the art. Alternatively, HGF or MSP and peptide fragments thereof, may be isolated from larger known prepropeptides that share a common or similar amino acid sequence.

Proteins and peptides derived from these and other sources, including manual or automated protein synthesis, may be quickly and easily tested for antiangiogenic activity using a biological activity assay such as the human umbilical vein endothelial cell proliferation assay (HUVEC) and the bovine capillary endothelial cell proliferation assay (BCE). Such assays are described in U.S. Pat. No. 5,639,725 which is incorporated herein by reference. Other bioassays for inhibiting activity include the chick CAM assay, the mouse corneal assay, and the effect of administering isolated or synthesized proteins on implanted tumors. The chick CAM assay is described by O'Reilly, et al. in “Angiogenic Regulation of Metastatic Growth” Cell, vol. 79 (2), Oct. 21, 1994, pp. 315-328, which is hereby incorporated by reference in its entirety. Applicants' invention also encompasses nucleic acid sequences that correspond to, and code for antiangiogenic kringle domain containing proteins and peptides, and to monoclonal and polyclonal antibodies that bind specifically to such protein molecules. The biologically active protein molecules, nucleic acid sequences corresponding to the proteins, and antibodies that bind specifically to the proteins of the present invention are useful for modulating angiogenic processes in vivo, and for diagnosing and treating endothelial cell-related diseases, for example by gene therapy.

Nucleic acid sequences that correspond to, and code for, kringle domain containing proteins and peptides such as HGF fragments and HGF fragment analogs, can be prepared based upon the knowledge of the amino acid sequence, and the art recognized correspondence between codons (sequences of three nucleic acid bases), and amino acids. Because of the degeneracy of the genetic code, wherein the third base in a codon may vary yet still code for the same amino acid, many different possible coding nucleic acid sequences are derivable for any particular protein or peptide fragment.

Nucleic acid sequences are synthesized using automated systems well known in the art. Either the entire sequence may be synthesized or a series of smaller oligonucleotides are made and subsequently ligated together to yield the full length sequence. Alternatively, the nucleic acid sequence may be derived from a gene bank using oligonucleotides probes designed based on the N-terminal amino acid sequence and well known techniques for cloning genetic material.

The present invention also encompasses gene therapy whereby genes encoding kringle domain containing proteins and peptides such as HGF or MSP fragments, are regulated in a patient. Various methods of transferring or delivering DNA to cells for expression of the gene product protein, otherwise referred to as gene therapy, are disclosed in Gene Transfer into Mammalian Somatic Cells in vivo, N. Yang, Crit. Rev. Biotechn. 12(4): 335-356 (1992). Gene therapy encompasses incorporation of DNA sequences into somatic cells or germ line cells for use in either ex vivo or in vivo therapy. Gene therapy functions to replace genes, augment normal or abnormal gene function, and to combat infectious diseases and other pathologies.

Strategies for treating these medical problems with gene therapy include therapeutic strategies such as identifying the defective gene and then adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene such as that for a desired HGF fragment may be placed in a patient and thus prevent occurrence of angiogenesis; or a gene that makes tumor cells more susceptible to radiation could be inserted and then radiation of the tumor would cause increased killing of the tumor cells.

Many protocols for transfer of kringle domain containing protein DNA, or corresponding regulatory sequences are envisioned in this invention. Transfection of promoter sequences, other than ones normally found specifically associated with such proteins or other sequences which would increase production of these proteins are also envisioned as methods of gene therapy. An example of this technology is found in Transkaryotic Therapies, Inc., of Cambridge, Mass., using homologous recombination to insert a “genetic switch” that turns on an erythropoietin gene in cells. See Genetic Engineering News, Apr. 15, 1994. Such “genetic switches” could be used to activate HGF (or HGF receptors) in cells not normally expressing HGF (or the HGF receptor).

Gene transfer methods for gene therapy fall into three broad categories: physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vitro gene transfer. In ex vivo gene transfer, cells are taken from the patient and grown in cell culture. The DNA is transfected into the cells, the transfected cells are expanded in number and then reimplanted in the patient. In in vitro gene transfer, the transformed cells are cells growing in culture, such as tissue culture cells, and not particular cells from a particular patient. These “laboratory cells” are transfected, the transfected cells are selected and expanded for either implantation into a patient or for other uses.

In vivo gene transfer involves introducing the DNA into the cells of the patient when the cells are within the patient. Methods include using virally mediated gene transfer using a noninfectious virus to deliver the gene in the patient or injecting naked DNA into a site in the patient and the DNA is taken up by a percentage of cells in which the gene product protein is expressed. Additionally, the other methods described herein, such as use of a “gene gun,” may be used for in vitro insertion of DNA encoding HGF kringle domains or HGF regulatory sequences.

Chemical methods of gene therapy may involve a lipid based compound, not necessarily a liposome, to ferry the DNA across the cell membrane.

Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the patient to provide the gene product from the inserted DNA.

Viral vectors have also been used to insert genes into cells using in vivo protocols. To direct tissue-specific expression of foreign genes, cis-acting regulatory elements or promoters that are known to be tissue specific can be used. Alternatively, this can be achieved using in situ delivery of DNA or viral vectors to specific anatomical sites in vivo. For example, gene transfer to blood vessels in vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression. In vivo gene transfer using retrovirus vectors has also been demonstrated in mammary tissue and hepatic tissue by injection of the altered virus into blood vessels leading to the organs.

Viral vectors that have been used for gene therapy protocols include but are not limited to, retroviruses, other RNA viruses such as poliovirus or Sindbis virus, adenovirus, adeno-associated virus, herpes viruses, SV 40, vaccinia and other DNA viruses. Replication-defective murine retroviral vectors are the most widely utilized gene transfer vectors. Murine leukemia retroviruses are composed of a single strand RNA complexed with a nuclear core protein and polymerase (pol) enzymes, encased by a protein core (gag) and surrounded by a glycoprotein envelope (env) that determines host range. The genomic structure of retroviruses include the gag, pol, and env genes enclosed at by the 5′ and 3′ long terminal repeats (LTR). Retroviral vector systems exploit the fact that a minimal vector containing the 5′ and 3′ LTRs and the packaging signal are sufficient to allow vector packaging, infection and integration into target cells providing that the viral structural proteins are supplied in trans in the packaging cell line. Fundamental advantages of retroviral vectors for gene transfer include efficient infection and gene expression in most cell types, precise single copy vector integration into target cell chromosomal DNA, and ease of manipulation of the retroviral genome.

The adenovirus is composed of linear, double stranded DNA complexed with core proteins and surrounded with capsid proteins. Advances in molecular virology have led to the ability to exploit the biology of these organisms to create vectors capable of transducing novel genetic sequences into target cells in vivo. Adenoviral-based vectors will express gene product peptides at high levels. Adenoviral vectors have high efficiencies of infectivity, even with low titers of virus. Additionally, the virus is fully infective as a cell free virion so injection of producer cell lines are not necessary. Another potential advantage to adenoviral vectors is the ability to achieve long term expression of heterologous genes in vivo.

Mechanical methods of DNA delivery include fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” and inorganic chemical approaches such as calcium phosphate transfection. Another method, ligand-mediated gene therapy, involves complexing the DNA with specific ligands to form ligand-DNA conjugates, to direct the DNA to a specific cell or tissue.

It has been found that injecting plasmid DNA into muscle cells yields high percentage of the cells which are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Particle-mediated gene transfer methods were first used in transforming plant tissue. With a particle bombardment device, or “gene gun,” a motive force is generated to accelerate DNA-coated high density particles (such as gold or tungsten) to a high velocity that allows penetration of the target organs, tissues or cells. Particle bombardment can be used in in vitro systems, or with ex vivo or in vivo techniques to introduce DNA into cells, tissues or organs.

Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. This technique can be used in in vitro systems, or with ex vivo or in vivo techniques to introduce DNA into cells, tissues or organs.

Carrier mediated gene transfer in vivo can be used to transfect foreign DNA into cells. The carrier-DNA complex can be conveniently introduced into body fluids or the bloodstream and then site specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoportein/polylysine conjugate system for carrying DNA to hepatocytes for in vivo gene transfer.

The transfected DNA may also be complexed with other kinds of carriers so that the DNA is carried to the recipient cell and then resides in the cytoplasm or in the nucleoplasm. DNA can be coupled to carrier nuclear proteins in specifically engineered vesicle complexes and carried directly into the nucleus.

Gene regulation of kringle domain containing proteins and peptides such as HGF kringle fragments may be accomplished by administering compounds that for example bind to HGF genes, or control regions associated with the HGF genes, or corresponding RNA transcript to modify the rate of transcription or translation. Additionally, cells transfected with a DNA sequence encoding HGF may be administered to a patient to provide an in vivo source of HGF fragments. For example, cells may be transfected with a vector containing a nucleic acid sequence encoding HGF. The term “vector” as used herein means a carrier that can contain or associate with specific nucleic acid sequences, which functions to transport the specific nucleic acid sequences into a cell. Examples of vectors include plasmids and infective microorganisms such as viruses, or non-viral vectors such as ligand-DNA conjugates, liposomes, lipid-DNA complexes. It may be desirable that a recombinant DNA molecule comprising HGF DNA sequence is operatively linked to an expression control sequence to form an expression vector capable of expressing HGF. The transfected cells may be cells derived from the patient's normal tissue, the patient's diseased tissue, or may be non-patient cells.

For example, tumor cells removed from a patient can be transfected with a vector capable of expressing HGF protein of the present invention, and re-introduced into the patient. The transfected tumor cells produce HGF levels in the patient that inhibit the growth of the tumor. Patients may be human or non-human animals. Cells may also be transfected by non-vector, or physical or chemical methods known in the art such as electroporation, ionoporation, or via a “gene gun.” Additionally, HGF DNA may be directly injected, without the aid of a carrier, into a patient. In particular, HGF DNA may be injected into skin, muscle or blood.

The gene therapy protocol for transfecting a kringle domain containing protein such as HGF into a patient may either be through integration of HGF DNA into the genome of the cells, into minichromosomes or as a separate replicating or non-replicating DNA construct in the cytoplasm or nucleoplasm of the cell. HGF expression may continue for a long-period of time or may be reinjected periodically to maintain a desired level of HGF protein in the cell, the tissue or organ or a determined blood level.

The present invention includes methods of treating or preventing angiogenic diseases and processes including, but not limited to, arthritis and tumors by stimulating the production of kringle domain containing proteins and peptides such as HGF fragments, and/or by administering substantially purified HGF fragments, or HGF fragment agonists or antagonists, and/or HGF fragment antisera to a patient. Additional treatment methods include administration of HGF fragments, HGF antisera, or HGF receptor agonists and antagonists linked to cytotoxic agents. It is to be understood that HGF can be animal or human in origin. HGF can also be produced synthetically by chemical reaction or by recombinant techniques in conjunction with expression systems. HGF, and fragments thereof, can also be produced by enzymatically cleaving different molecules, including HGF precursors, containing sequence homology or identity with segments of HGF to generate peptides having anti-angiogenesis activity.

The present invention further includes methods of treating or preventing angiogenic diseases and processes including, but not limited to, arthritis and tumors by stimulating the production of kringle domain containing proteins and peptides such as MSP fragments, and/or by administering substantially purified MSP fragments, or MSP fragment agonists or antagonists, and/or HGF fragment antisera to a patient. Additional treatment methods include administration of HGF, MSP fragments, MSP antisera, or MSP receptor agonists and antagonists linked to cytotoxic agents. It is to be understood that MSP can be animal or human in origin. MSP can also be produced synthetically by chemical reaction or by recombinant techniques in conjunction with expression systems. MSP, and fragments thereof, can also be produced by enzymatically cleaving different molecules, including MSP precursors, containing sequence homology or identity with segments of MSP to generate peptides having anti-angiogenesis activity.

Antibodies that specifically bind kringle domain containing proteins and peptides such as HGF fragments can be employed to modulate endothelial-dependent processes such as reproduction., development, and wound healing and tissue repair. In addition, antisera directed to the Fab regions of HGF antibodies for example can be administered to block the ability of endogenous HGF antisera to bind HGF fragments.

Antibodies specific kringle domain containing proteins and peptides such as HGF fragments, and HGF fragment analogs, are made according to techniques and protocols well known in the art. The antibodies may be either polyclonal or monoclonal. The antibodies are utilized in well know immunoassay formats, such as competitive and non-competitive immunoassays, including ELISA, sandwich immunoassays and radioimmunoassays (RIAs), to determine the presence or absence of the endothelial proliferation inhibitors of the present invention in body fluids. Examples of body fluids include but are not limited to semen, blood, serum, peritoneal fluid, pleural fluid, cerebrospinal fluid, uterine fluid, saliva, and mucus.

The proteins, nucleic acid sequences and antibodies of the present invention are useful for diagnosing and treating endothelial cell-related diseases and disorders. A particularly important endothelial cell process is angiogenesis, the formation of blood vessels. Angiogenesis-related diseases may be diagnosed and treated using the endothelial cell proliferation inhibiting proteins of the present invention. Angiogenesis-related diseases include, but are not limited to, angiogenesis-dependent cancer, including, for example, solid tumors, blood born tumors such as leukemias, and tumor metastases; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis blindness; plaque neovascularization; telangiectasia; hemophiliac joints; angiofibroma; and wound granulation. The endothelial cell proliferation inhibiting proteins of the present invention are useful in the treatment of disease of excessive or abnormal stimulation of endothelial cells. These diseases include, but are not limited to, intestinal adhesions, atherosclerosis, scleroderma, and hypertrophic scars, i.e., keloids. They are also useful in the treatment of diseases that have angiogenesis as a pathologic consequence such as cat scratch disease (Rochele minalia quintosa) and ulcers (Helicobacter pylorii).

The angiogenic regulating proteins of the present invention can be used as a birth control agent by reducing or preventing uterine vascularization required for embryo implantation. Thus, the present invention provides an effective birth control method when an amount of a kringle domain containing protein composition comprising for example inhibitory HGF fragments sufficient to prevent embryo implantation is administered to a female. In one aspect of the birth control method, an amount of the inhibiting protein sufficient to block embryo implantation is administered before or after intercourse and fertilization have occurred, thus providing an effective method of birth control, possibly a “morning after” method. While not wanting to be bound by this statement, it is believed that inhibition of vascularization of the uterine endometrium interferes with implantation of the blastocyst. Similar inhibition of vascularization of the mucosa of the uterine tube interferes with implantation of the blastocyst, preventing occurrence of a tubal pregnancy. Administration methods may include, but are not limited to, pills, injections (intravenous, subcutaneous, intramuscular), suppositories, vaginal sponges, vaginal tampons, and intrauterine devices. It is also believed that administration of the anti-angiogenic compositions of the present invention will interfere with normal enhanced vascularization of the placenta, and also with the development of vessels within a successfully implanted blastocyst and developing embryo and fetus.

Conversely, blockade of kringle domain containing protein receptors with corresponding analogs which act as receptor antagonists, may promote angiogenic activity such as endothelialization and vascularization. Such effects may be desirable in situations of inadequate vascularization of the uterine endometrium and associated infertility, wound repair, healing of cuts and incisions, treatment of vascular problems in diabetics, especially retinal and peripheral vessels, promotion of vascularization in transplanted tissue including muscle and skin, promotion of vascularization of cardiac muscle especially following transplantation of a heart or heart tissue and after bypass surgery, promotion of vascularization of solid and relatively avascular tumors for enhanced cytotoxin delivery, and enhancement of blood flow to the nervous system, including but not limited to the cerebral cortex and spinal cord.

The present invention also relates to methods of using angiogenic peptide fragments of kringle domain containing proteins and peptides such as HGF, nucleic acid sequences corresponding to HGF fragments, and antibodies that bind specifically to HGF fragments and related peptides, to diagnose endothelial cell-related diseases and disorders.

The invention further encompasses a method for identifying kringle domain containing protein-specific receptors, and the receptor molecules identified and isolated thereby. The present invention also provides a method for quantitation of such receptors.

A particularly important aspect of the present invention is administration of HGF fragments either alone or in combination with one or more anti-angiogenic agents, such as ENDOSTATIN protein, ANGIOSTATIN protein, or METASTATIN™ protein (Entremed, Inc., Rockville, Md.), in an amount sufficient to inhibit tumor growth and cause sustainable regression of tumor mass to microscopic size. Accordingly, the present invention also includes formulations effective for treating or curing angiogenesis-dependent cancers and tumors.

More particularly, recombinant HGF fragments, from insect cells or E. coli, for example, can potently inhibit angiogenesis and the growth of metastases. It is contemplated as part of the present invention that HGF fragments can be isolated from a body fluid such as semen, blood or urine of patients, or that HGF fragments can be produced by recombinant DNA methods or synthetic peptide chemical methods that are well known to those of ordinary skill in the art. Protein purification methods are well known in the art.

One example of a method of producing a desired kringle domain containing protein such as HGF fragments using recombinant DNA techniques entails the steps of (1) identifying an HGF fragment as discussed above, and as more fully described below, (2) synthetically generating a DNA oligonucleotide probe that corresponds to the protein sequence, (3) conducting PCR from human liver cDNA (4) inserting the gene into an appropriate vector such as an expression vector, (5) inserting the gene-containing vector into a microorganism or other expression system capable of expressing the inhibitor gene, and (6) isolating the recombinantly produced inhibitor. The above techniques are more fully described in laboratory manuals such as “Molecular Cloning: A Laboratory Manual” Latest Edition by Sambrook et al., Cold Spring Harbor Press, 1989.

Yet another method of producing desired proteins of the present invention such as HGF fragments is by peptide synthesis. For example, once a biologically active fragment of HGF is found, it can be sequenced, for example by automated peptide sequencing methods. Alternatively, once the gene or DNA sequence which codes for HGF fragment is isolated, for example by the methods described above, the DNA sequence can be determined, which in turn provides information regarding the amino acid sequence. Thus, if the biologically active fragment is generated by specific methods, such as tryptic digests, or if the fragment is N-terminal sequenced, the remaining amino acid sequence can be determined from the corresponding DNA sequence.

Once the amino acid sequence of the peptide is known, for example the N-terminal 20 amino acids, the fragment can be synthesized by techniques well known in the art, as exemplified by “Solid Phase Peptide Synthesis: A Practical Approach” E. Atherton and R. C. Sheppard, IRL Press, Oxford England. Similarly, multiple fragments can be synthesized which are subsequently linked together to form larger fragments. These synthetic peptide fragments can also be made with amino acid substitutions at specific locations in order to test for agonistic and antagonistic activity in vitro and in vivo.

The synthetic peptide fragments of kringle domain containing proteins and peptides such as HGF have a variety of uses. The peptide that binds to the HGF receptor with high specificity and avidity is radiolabeled and employed for visualization and quantitation of binding sites using autoradiographic and membrane binding techniques. Knowledge of the binding properties of the HGF receptor facilitates investigation of the transduction mechanisms linked to the receptor.

Different peptide fragments of the intact HGF molecule can be synthesized for use in several applications including, but not limited to the following; as antigens for the development of specific antisera, as agonists and antagonists active at HGF binding sites, as peptides to be linked to cytotoxic agents for targeted killing of cells that bind HGF. The amino acid sequences that comprise these peptides are selected on the basis of their position on the exterior regions of the molecule and are accessible for binding to antisera. Peptides can be synthesized in a standard microchemical facility and purity checked with HPLC and mass spectrophotometry. Methods of peptide synthesis, HPLC purification and mass spectrophotometry are commonly known to those skilled in these arts.

HGF kringle containing fragments or peptides can also be produced in recombinant E. coli, or in insect or yeast expression systems, mammalian cell expression systems and transgenic expression systems and purified with column chromatography.

HGF peptides can be chemically coupled to isotopes, enzymes, carrier proteins, cytotoxic agents, fluorescent molecules and other compounds for a variety of applications. The efficiency of the coupling reaction is determined using different techniques appropriate for the specific reaction.

Systematic substitution of amino acids within the synthesized peptides yields high affinity peptide agonists and antagonists to HGF receptors that enhance or diminish HGF binding to its receptor. Such agonists are used to suppress the growth of primary and metastatic tumors, thereby limiting the spread of cancer. Antagonists to HGF are applied in situations of inadequate vascularization, to block the inhibitory effects of HGF and possibly promote angiogenesis. This treatment may have therapeutic effects to promote wound healing in diabetics.

HGF peptides are employed to develop affinity columns for isolation of the HGF receptor from cultured cells. Isolation and purification of the HGF receptor is followed by amino acid sequencing. Next, nucleotide probes are developed for insertion into vectors for expression of the receptor. These techniques are well known to those skilled in the art. These techniques can be helpful in defining minimal structures of HGF for receptor engagement.

Cytotoxic agents, such as ricin, are linked to the kringle domain containing proteins and peptides of the present invention such as HGF kringle fragments and high affinity HGF peptide fragments, thereby providing a tool for destruction of cells that bind HGF. These cells may be found in many locations, including but not limited to, metastases and primary tumors. Peptides linked to cytotoxic agents are infused in a manner designed to maximize delivery to the desired location. For example, ricin-linked high affinity HGF fragments are delivered through a cannula into vessels supplying the target site or directly into the target. Such agents are also delivered in a controlled manner through osmotic pumps coupled to infusion cannulae. A combination of HGF antagonists may be co-applied with stimulators of angiogenesis to increase vascularization of tissue.

Antiserum against kringle domain containing proteins and peptides such as HGF fragments can be generated. After peptide synthesis and purification, both monoclonal and polyclonal antisera are raised using established techniques known to those skilled in the art. For example, polyclonal antisera may be raised in rabbits, sheep, goats or other animals. HGF peptides conjugated to a carrier molecule such as bovine serum albumin, are combined with an adjuvant mixture, emulsified and injected subcutaneously at multiple sites on the back, neck, flanks, and sometimes in the footpads. Booster injections are made at regular intervals, such as every 2 to 4 weeks. Blood samples are obtained by venipuncture, for example using the marginal ear veins after dilation, approximately 7 to 10 days after each injection. The blood samples are allowed to clot overnight at 4° C. and are centrifuged at approximately 2400×g at 4° C. for about 30 minutes.

All serum samples from generation of polyclonal antisera or media samples from production of monoclonal antisera are analyzed for determination of titer. Titer is established through several means, for example, using dot blots and density analysis, and also with precipitation of radiolabeled peptide-antibody complexes using protein A, secondary antisera, cold ethanol or charcoal-dextran followed by activity measurement with a gamma counter. The highest titer antisera are also purified on affinity columns which are commercially available. HGF peptides are coupled to the gel in the affinity column. Antiserum samples are passed through the column and anti-HGF fragment antibodies remain bound to the column. These antibodies are subsequently eluted, collected and evaluated for determination of titer and specificity.

The highest titer HGF fragment antisera is tested to establish the following; a) optimal antiserum dilution for highest specific binding of the antigen and lowest non-specific binding, b) the ability to bind increasing amounts of HGF peptide in a standard displacement curve, c) potential cross-reactivity with related peptides and proteins, including HGF related species, d) ability to detect HGF peptides in extracts of, semen, plasma, urine, tissues, and in cell culture media.

According to the present invention, kringle domain containing protein compositions such as HGF fragment compositions, may be used in combination with other compositions and procedures for the treatment of diseases. For example, a tumor may be treated conventionally with surgery, radiation or chemotherapy combined with or without HGF fragment compositions and then such compositions may be subsequently administered to the patient to extend the dormancy of micrometastases and to stabilize any residual primary tumor.

It is to be understood that the present invention is contemplated to include any derivatives of kringle domain containing proteins and peptides that have angiogenic activity. The present invention includes the entire HGF protein including the kringle domains, derivatives of the HGF protein and biologically-active fragments of the HGF protein. These include proteins with HGF activity that have amino acid substitutions or have sugars or other molecules attached to amino acid functional groups. The present invention also includes genes that code for HGF and HGF receptors, HGF kringle domains, and to proteins that are expressed by those genes.

The present invention includes the entire MSP protein, derivatives of the MSP protein, MSP kringle fragments, and biologically-active fragments of the MSP protein. These include proteins with MSP activity that have amino acid substitutions or have sugars or other molecules attached to amino acid functional groups. The present invention also includes genes that code for MSP and MSP receptors as well as MSP kringle domain fragments, and to proteins that are expressed by those genes.

The kringle domain containing protein having antiangiogenic activity described above can be provided as isolated and substantially purified proteins and protein fragments in pharmaceutically acceptable formulations using formulation methods known to those of ordinary skill in the art. These formulations can be administered by standard routes. In general, the combinations may be administered by the topical, transdermal, intraperitoneal, intracranial, intracerebroventricular, intracerebral, intravaginal, intrauterine, oral, rectal or parenteral (e.g., intravenous, intraspinal, subcutaneous or intramuscular) route. In addition, the proteins may be incorporated into biodegradable polymers allowing for sustained release of the compound, the polymers being implanted in the vicinity of where drug delivery is desired, for example, at the site of a tumor or implanted so that the HGF is slowly released systemically. Osmotic minipumps may also be used to provide controlled delivery of high concentrations of HGF kringle fragments through cannulae to the site of interest, such as directly into a metastatic growth or into the vascular supply to that tumor. The biodegradable polymers and their use are described, for example, in detail in Brem et al., J. Neurosurg. 74:441-446 (1991).

The formulations of the present invention include those suitable for oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

The dosage of the compositions of the present invention will depend on the disease state or condition being treated and other clinical factors such as weight and condition of the human or animal and the route of administration of the compound. For example, for treating humans or animals, between approximately 0.5 to 500 mg/kilogram is typical broad range for administering a HGF protein, or a composition comprising kringle domain fragments of HGF. Depending upon the half-life of the protein in the particular animal or human, the protein can be administered between several times per day to once a week. It is to be understood that the present invention has application for both human and veterinary use. The methods of the present invention contemplate single as well as multiple administrations, given either simultaneously or over an extended period of time.

Preferred unit dosage formulations are those containing a daily dose or unit, daily sub-dose, as herein above recited, or an appropriate fraction thereof, of the administered ingredient. It should be understood that in addition to the ingredients, particularly mentioned above, the formulations of the present invention may include other agents conventional in the art having regard to the type of formulation in question.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

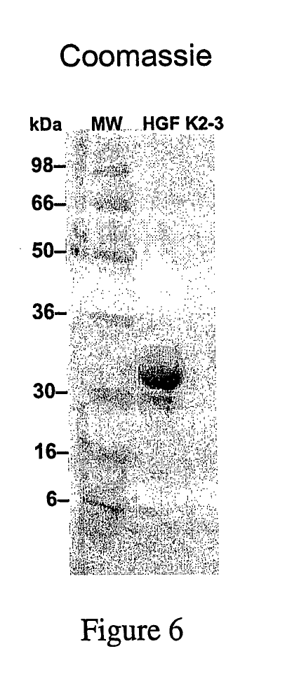

Purified Human Recombinant HGF K2-3 Expressed in Pichia pastori (FIG. 6)

The HGF K2-3 gene was cloned into pPICZaA and expressed in X33 P. pastoris strain. The fermentation supernatant was purified through ion exchange and hydrophobic interaction chromatography. 11 μg of purified HGF K2-3 was analyzed on the denaturing and non-reducing SDS-PAGE gel stained with Coomassie blue.

### EXAMPLE 2

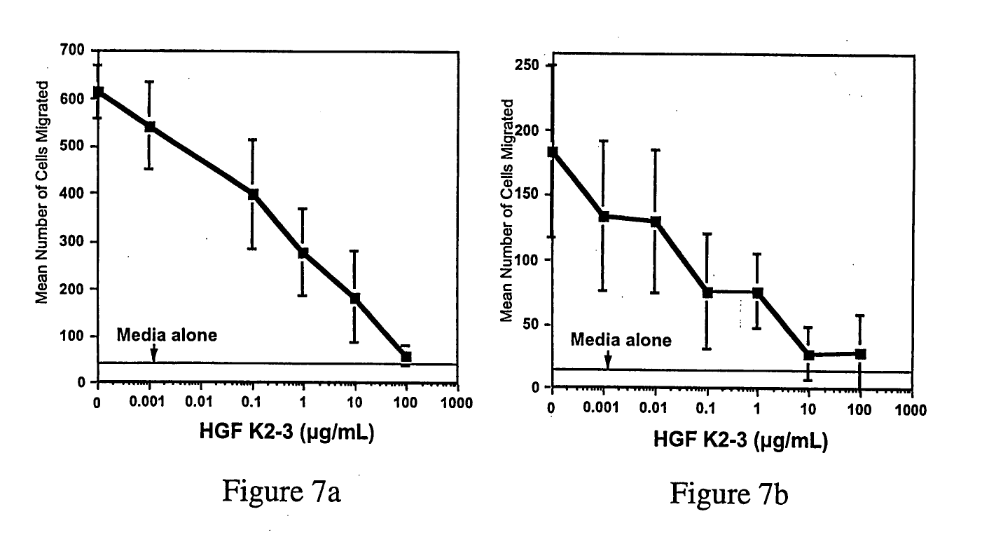

HGF K2-3 Inhibits VEGF as Well as HGF Stimulated HUVEC Migration. (FIGS. 7a and 7b)

Micro Chemotaxis Assay

Apparatus:

Chamber: Neuro Probe Standard 48 Well Chemotaxis Chamber Cat # AP48 (301-229-8598)

Filter Membrane: Poretics Membrane Polycarbonate PVP Free 8 micron 25×80 mm (Osmonics Inc.800-444-8212 Cat #10474)

Forceps: VWR Scientific 800-234-9300 Cat# 25681-269. For ease of handling membranes use these forceps.

Preparation of Filter Membrane:

Coat membranes with Rat tail Collagen Type 1 (BD Collaborative Res Cat # 40236) at 100 mg/ml in 29 ml of 0.2N Acetic Acid. For 0.2N Acetic Acid add 345 ml of Glacial Acetic Acid (99%) in 29.66 sterile H20 (BRL).

1. Dilute collagen (100 mg/ml) in 0.2N Acetic Acid in 50 ml conical tube. Mix well.

2. Place collagen solution in either a petri dish or small plastic staining box. Submerge membranes individually (10 membranes).

3. Agitate slowly on rocker at room temperature for 48 hours.

4. Air dry filters in laminar flow hood by laying out filters on open petri dishes.

5. Store dry filters in covered container at room temperature (2 weeks).

Assay:

Assay Media: Medium −200 supplemented with 1% L-glutamine (BioWhittaker) and 0.1% BSA (Sigma Cat# A8412)

1. Harvest HUVEC p2-p7 from flasks by trypsinization. Neutralize trypsin with growth medium. Remove trypsin/versene/media by centrifugation at 1000 rpm for 5 minutes. Resuspend in 10 ml of assay media. Do not over trypsinize cells or over centrifuge cells.

2. Count cells by dilution with Trypan Blue solution. Determine viability. Resuspend cells at 2×105/ml in assay media.

3. Before adding cells to chambers, preincubate cells with test proteins in 17×100 mm (14 ml) polypropylene round bottom tube (Falcon 35-2059) for 30 minutes at 37° C. with 5% CO₂. Do not snap top of tube. Incubate control cells with assay media.

4. Approximately 5 minutes before addition of cells prepare chamber. To the first three columns of the bottom chamber add approximately 28 ml of assay media alone. This volume varies with individual chambers from 25-30 ml. Adjust volume so that a slight positive meniscus is formed over well. Fill rest of wells with chemoattractant (e.g., VEGF165 R&D Cat #293-VE) 2-10 ng/ml. Reconstitute VEGF in assay media. Optimum concentration depends on cells. The last three columns are stimulated controls.

5. All manipulations with membranes are done with forceps. Cut off small corner of membrane and orient cut off corner with NP trademark. Place membrane on top of lower chamber shiny side up. Do not adjust membrane as this will contaminate wells.

6. Gently place silicone gasket on top of membrane. Then place the top portion of the chamber oriented with the trademark. With firm even pressure screw on the thumb nuts tightly. Note: Cells will leak if this is not done correctly.

7. Add 50 ml of either control cells or treated cells to upper chambers. To avoid bubbles lift pipette tip while adding cells. Be careful not to puncture membrane.

8. Place chamber in 150 mm petri dish with moist paper towel. Incubate chamber or 6 h at 37° C. with 5% CO₂.

9. After 6 hours, gently remove thumb nuts and upper chamber. Peel off membrane which is stuck to the gasket with forceps.

10. Fix and stain filter membrane with Diff-Quik (Dade Int. Cat# 84132-10) Fix for 2 minutes; Solution I for 2 minutes; Solution II for 3 minutes.

11. Rinse membrane 2×in distilled H2O. Place membrane on top of 3×2 glass microscope slide (VWR Cat # 28351-100). With a wet Kim-Wipe remove non-migrating cells while holding onto membrane to prevent movement. After removal of cells allow membrane to dry. Place 4 small drops of super-glue (use pipette tip on each corner of the slide. Place additional slide on top. View under microscope to determine total number of migrated cells.

The migration of HUVECs was evaluated with the micro chemotaxis chamber (modified Boyden chamber, Neuro Probe, Inc., Gaithersburg, Md.) described above which allows for the measurement of cell movement and directionality. HUVECs pretreated with HGF K2-3 or basal media (M−200 containing 0.1% BSA) for 30′ at 37° C. were placed into the upper chamber. The lower chamber contained basal media with (a) VEGF (5 ng/ml) or (b) HGF (50 ng/mL) or without. Cells were allowed to migrate through an 8 mm polycarbonate PVP free filter coated with 100 μg/ml of rat tail collagen type 1 (Collaborative Biomedical Products, Bedford, Mass.). The chamber was then incubated as above for 6 h. The non-migrated cells were removed and the filter was fixed and stained with Diff-Quik (Dade Diagnostics, Aquado, Puerto Rico). The numbers of migrated cells were determined using the Image-Pro Plus analysis system (Media Cybernetics, Silver Spring, Md.). BSA at 100 μg/ml showed no inhibition effect on the growth factor stimulated HUVEC migration (data not shown).

### EXAMPLE 3

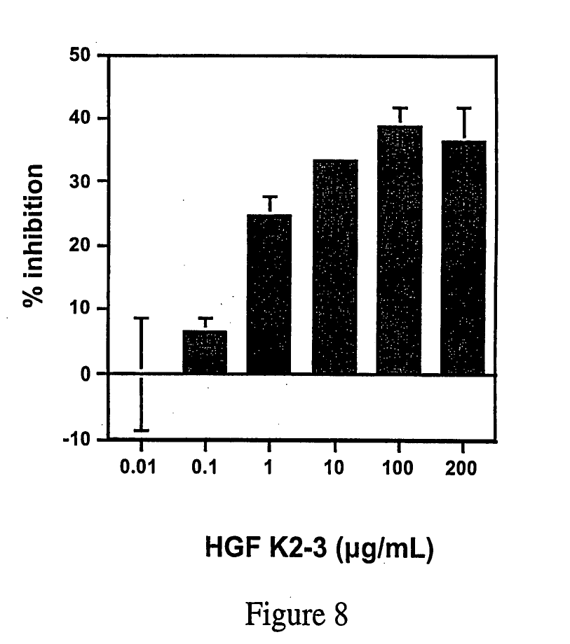

HGF K2-3 Inhibits HUVEC Tube Formation. (FIG. 8)

HUVECs were trypsinized and resuspend at 1×10 5 cells/ml in the assay media [medium −200 supplemented with LSGS and 5% heat inactivated FBS (Hyclone)]. 100 ml of HGFK2-3 dilution in assay and 100 ul of HUVECs were plated with onto a Matrigel substratum(Collaborative Biomedical Products, Bedford, Mass.) for 16 h at 37° C. After incubation, endothelial cells were examined microscopically and evaluated for tube formation by counting the number of junctions. BSA at 200 μg/ml showed no inhibition effect (data not shown).

### EXAMPLE 4

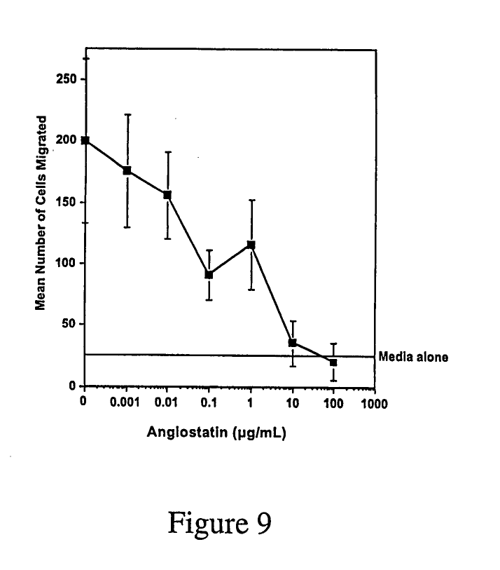

Angiostatin Protein Inhibits HGF Stimulated HUVEC Migration. (FIG. 9)

The migration of HUVECs was evaluated with a micro chemotaxis chamber (modified Boyden chamber, Neuro Probe, Inc., Gaithersburg, N. Dak.). HUVECs pretreated with Angiostatin protein or basal media (M−200 containing 0.1% BSA) for 30′ at 37° C. were placed into the upper chamber. The lower chamber contained basal media with HGF (50 ng/ml) or without. Cells were allowed to migrate through an 8 mm polycarbonate PVP free filter coated with 100 mg/ml of rat tail collagen type 1 (Collaborative Biomedical Products, Bedford, Mass.). The chamber was then incubated as above for 6 h. The non-migrated cells were removed and the filter was fixed and stained with Diff-Quik (Dade Diagnostics, Aquado, Puerto Rico). The numbers of migrated cells were determined using the Image-Pro Plus analysis system (Media Cybernetics, Silver Spring, Md.). BSA at 100 ug/ml showed no inhibition effect on the growth factor stimulated HUVEC migration (data not shown).

### EXAMPLE 5

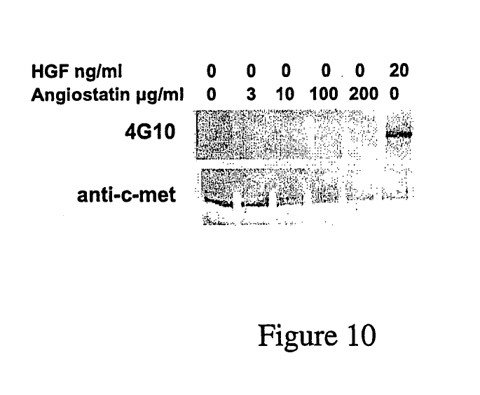

### Angiostatin Protein Does Not Induce c-met Phosphorylation. (FIG. 10)

A549 cells were starved in serum free media plus 0.1% BSA for 6 hours and then treated with Angiostatin protein or HGF for 10 min. at 37° C. The cells were lysed and the supernatant was then immunoprecipated with 15 ul of rabbit anti-c-met antibodies and analyzed with Western blots probed with anti-phosphotyrosine antibody (4G10) or anti-c-met antibodies.

### EXAMPLE 6

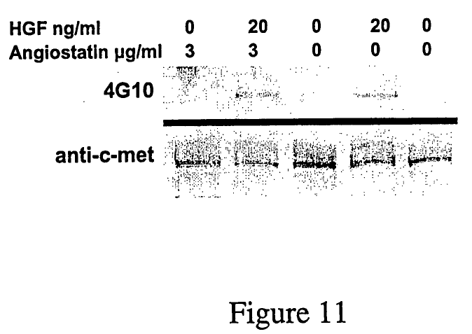

### Angiostatin Protein Does Not Inhibit HGF Stimulated C-met Phosphorylation. (FIG. 11)

The HUVECs were starved in serum free media plus 0.1% BSA for 6 hours and then treated with Angiostatin protein at 4° C. for 30 min. The cold media was replaced with fresh media (37° C.) with the same concentration of Angiotstatin and then induced with 20 ng/ml HGF for 10 min. at 37° C. The cells were lysed and the supernatant was then immunoprecipated with 15 ul of rabbit anti-c-met anti-bodies and analyzed with Western blots probed with anti-phosphotyrosine antibody (4G10) or anti-c-met antibodies.

We claim:

1. A method of inhibiting angiogenesis in an animal comprising administering to the animal an angiogenesis inhibiting amount of a composition comprising kringle domain-containing proteins and peptides.

2. The method of claim 1, wherein kringle domain containing proteins and peptides comprise hepatocyte growth factor, macrophage stimulating protein, and biologically active fragments thereof.

3. The method of claim 2, wherein the hepatocyte growth factor has the amino acid sequence as set forth in SEQ ID NO: 1, or an antiangiogenic fragment thereof.

4. The method of claim 1, further comprising a pharmaceutically acceptable excipient.

5. The method of claim 1, wherein the composition further comprises the peptide having the amino acid sequence set forth in SEQ ID NO: 2.

6. The method of claim 1, wherein the composition further comprises the peptide having the amino acid sequence set forth in SEQ ID NO: 3.

7. The method of claim 1, wherein the animal has an angiogenesis-mediated disease selected from the group consisting of angiogenesis-dependent cancers, benign tumors, rheumatoid arthritis, psoriasis, ocular angiogenesis diseases, Osler-Webber Syndrome, myocardial angiogenesis, plaque neovascularization, telangiectasia, hemophiliac joints, angiofibroma, wound granulation, intestinal adhesions, atherosclerosis, scleroderma, hypertrophic scars, cat scratch disease and Helicobacter pylori ulcers.

8. A method of inhibiting cell proliferation comprising, administering to a cell undergoing proliferation a proliferation inhibiting amount of a composition comprising biologically active fragments of HGF to inhibit cell proliferation.

9. The method of claim 8, wherein the cell proliferation comprises endothelial cell proliferation and smooth muscle cell proliferation.

10. The method of claim 8, wherein the kringle domain containing proteins and peptides comprise hepatocyte growth factor, macrophage stimulating protein, and biologically active fragments thereof.

11. The method of claim 8, wherein the hepatocyte growth factor has the amino acid sequence set forth in SEQ ID NO: 1, or an antiproliferative fragment thereof.

12. The method of claim 11, wherein the composition further comprises the peptide having the amino acid sequence set forth in SEQ ID NO: 2.

13. The method of claim 11, wherein the composition further comprises the peptide having the amino acid sequence set forth in SEQ ID NO: 3.

14. The method of claim 11, wherein the cell proliferation is related to an angiogenesis-mediated disease.

15. The method of claim 14, wherein the angiogenesis-mediated disease is selected from the group consisting of angiogenesis-dependent cancers, benign tumors, rheumatoid arthritis, psoriasis, ocular angiogenesis diseases, Osler-Webber Syndrome, myocardial angiogenesis, plaque neovascularization, telangiectasia, hemophiliac joints, angiofibroma, wound granulation, intestinal adhesions, atherosclerosis, scleroderma, hypertrophic scars, cat scratch disease and Helicobacter pylori ulcers.

16. A method of diagnosing a disease or determining the prognosis of a disease mediated by angiogenesis comprising obtaining a biological sample and determining the levels of HGF in the sample.

17. The method of claim 16, wherein the kringle domain containing proteins and peptides comprise hepatocyte growth factor, macrophage stimulating protein and biologically active fragments thereof.

18. The method of claim 17 wherein the hepatocyte growth factor comprises the amino acid sequence as set forth in SEQ ID NO: 1, or an anti-angiogenic fragment thereof.

19. The method of claim 17 wherein the macrophage stimulating protein comprises the amino acid sequence as set forth in SEQ ID NO: 4, or an anti-angiogenic fragment thereof.

20. The method of claim 16, wherein the angiogenesis-mediated disease is selected from the group consisting of angiogenesis-dependent cancers, benign tumors, rheumatoid arthritis, psoriasis, ocular angiogenesis diseases, Osler-Webber Syndrome, myocardial angiogenesis, plaque neovascularization, telangiectasia, hemophiliac joints, angiofibroma, wound granulation, intestinal adhesions, atherosclerosis, scleroderma, hypertrophic scars, cat scratch disease and Helicobacter pylori ulcers.
